(19) United States
(12) Patent Application Publication
(10) Pub. No.: US 2008/0108983 A1
(43) Pub. Date: May 8, 2008

(54) Dual Core Optic Fiber Illuminated Laser Probe

(75) Inventor: Timothy J. Nadolski, Webster Groves, MO (US)

Correspondence Address: THOMPSON COBURN, LLP, ONE US BANK PLAZA, SUITE 3500, ST LOUIS, MO, 63101, US

(73) Assignee: SYNERGETICS, INC., St. Charles, MO (US)

(21) Appl. No.: 11/557,264

(22) Filed: Nov. 7, 2006

(51) Int. Cl. A61B 18/18

(52) U.S. Cl. 606/16

(57) ABSTRACT

A microsurgical laser probe primarily used in ophthalmic surgery provides both laser light and illumination light to a surgical site from a single light source. The laser probe has a dual core optic fiber that transmits both laser light and illumination light to the surgical site. A center core of the optic fiber transmits the laser light through the optic fiber and emits the laser light at the surgical site. The center core of the fiber is surrounded by an outer fiber core. The outer fiber core has an interior bore that contains the center core optic fiber. The outer fiber core transmits illumination light through the optic fiber and emits the illumination light at the surgical site.

### BACKGROUND OF THE INVENTION

(1) Field of the Invention

The present invention pertains to a microsurgical laser probe used primarily in ophthalmic surgery where the probe provides both laser light and illumination light to a surgical site. More specifically, the laser probe of the invention has a dual core optic fiber that transmits both laser light and illumination light to a surgical site. A center core of the optic fiber transmits the laser light through the fiber and emits the laser light at the surgical site. The center core of the fiber is surrounded by an outer fiber core. The outer core transmits illumination light through the fiber and emits the illumination light at the surgical site.

(2) Description of the Related Art

Laser endoprobes or microsurgical probes and white light illumination probes have been employed in performing ophthalmic surgery procedures for many years. Until the early 1980's, laser probes and illumination probes were separate and independent instruments. Examples of these are disclosed in the U.S. Pat. No. 7,060,028 and U.S. Pat. No. 4,607,622.

In 1986, the Mir Ali U.S. Pat. No. 4,583,526 disclosed a handpiece that had a carbon dioxide laser and an illumination light source traveling in parallel. The U.S. Patents of Easley et al. U.S. Pat. No. 5,275,593 and U.S. Pat. No. 5,356,407 also disclose instruments that combined both laser and illumination fibers in the same probe. Presently, almost all of the major ophthalmic surgery instrument manufacturing companies have a line of illuminated laser probes. These probes work on the basic premise that two or more fibers are fed down the length of a tubular tip at the front of an instrument. The distal ends of the fibers are positioned adjacent to the distal end of the tip. The proximal end of one of the fibers is connected to a laser light source, and the proximal end of the other fiber is connected to an illumination light source.

As ophthalmic illumination sources have improved, the size and the number of optic fibers that are used in microsurgical instruments has decreased. In 2004, Synergetics, Inc. developed a light source capable of coaxially aligning a laser light and a white light illumination path so that both would be able to travel down a single fiber. This allowed for the use of a single optic fiber that would simultaneously provide both illumination light and laser light at the surgical site.

However, there were problems associated with using a single optic fiber for the transmission of both illumination light and laser light to the surgical site. It was observed that the light emitted from the optic fiber distal end would have the same divergence angle as the light delivered by the light source to the optic fiber proximal end. This meant that the area of illumination at the surgical site would be directly proportional to the size of the laser light spot at the surgical site. For example, the illumination light divergence angle at the distal end of the optic fiber would normally be 30 degrees off the center axis, and the laser light divergence angle at the distal end of the optic fiber would normally be 8 degrees off the axis. When the microsurgical probe distal end tip would be positioned close enough to the surgical site to get a laser light spot sized small enough for a desired burn, the area of illumination would be very small.

Illuminated laser probes have been designed according to two methods to compensate for this shortcoming. Probes have been designed with two staggered optic fibers, with the distal end of the illumination optic fiber being spaced back from the distal end of the laser optic fiber. This design would provide a larger area of illumination at the surgical site, but would produce a shadow in the illumination area where the illumination light is blocked by the distal end of the laser optic fiber. The other solution was to make the illumination optic fiber a wide field fiber. This was done through the use of a cone-shaped lens such as that disclosed in the U.S. Pat. No. 6,829,411, or the use of optical films, or an emulsion of glass spheres or balls. However, each of these would also produce a shadow of the laser optic fiber. Furthermore, the single optic fiber design would not allow for any of these options because it would scatter all of the illumination light and laser light transmitted equally.

### BRIEF DESCRIPTION OF THE DRAWINGS

The dual core fiber optic laser probe of the invention overcomes the disadvantages of prior art illuminated laser probes. The features of the invention that overcome the shortcomings of the prior art are set forth in the following detailed description of the preferred embodiments of the invention and in the drawing figures.

FIG. 1 is a cross-section view of the surgical instrument of the invention.

FIG. 2 is an enlarged partial view of the distal end of the instrument shown in FIG. 1.

FIG. 3 is an enlarged partial view of the proximal end of the instrument shown in FIG. 1.

FIG. 4 is an enlarged cross-section view of the dual core optic fiber of the instrument of the invention.

FIG. 5 is an enlarged schematic representation of the proximal end of the dual core optic fiber.

FIGS. 6 and 7 are enlarged schematic representations of the distal ends of two different embodiments of the dual core optic fiber.

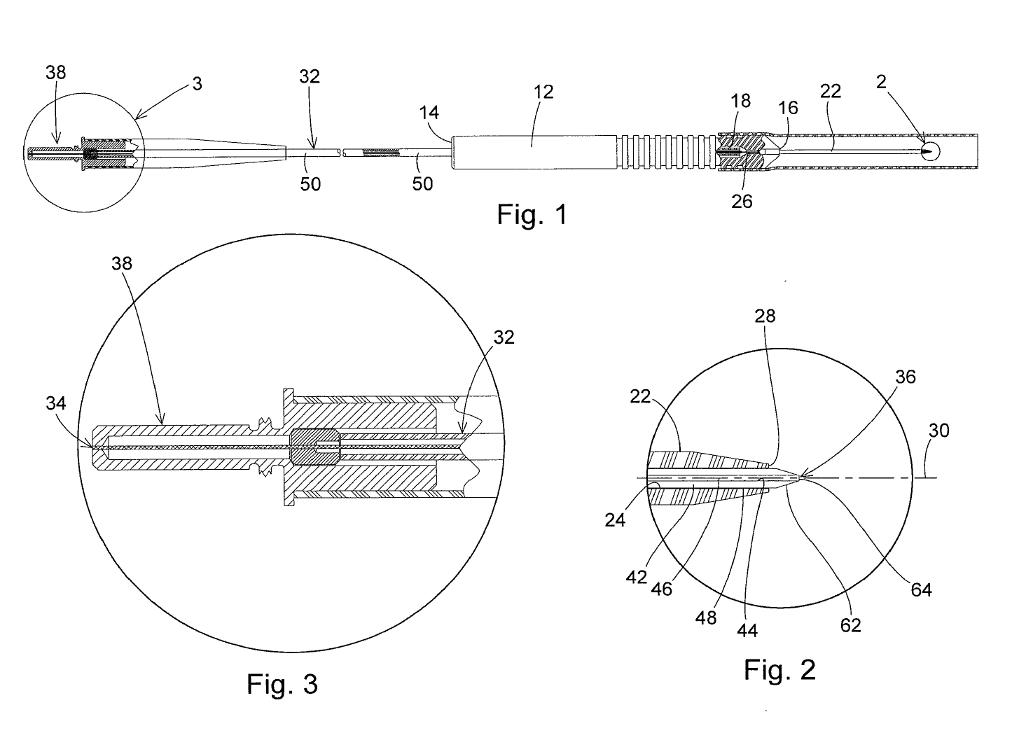

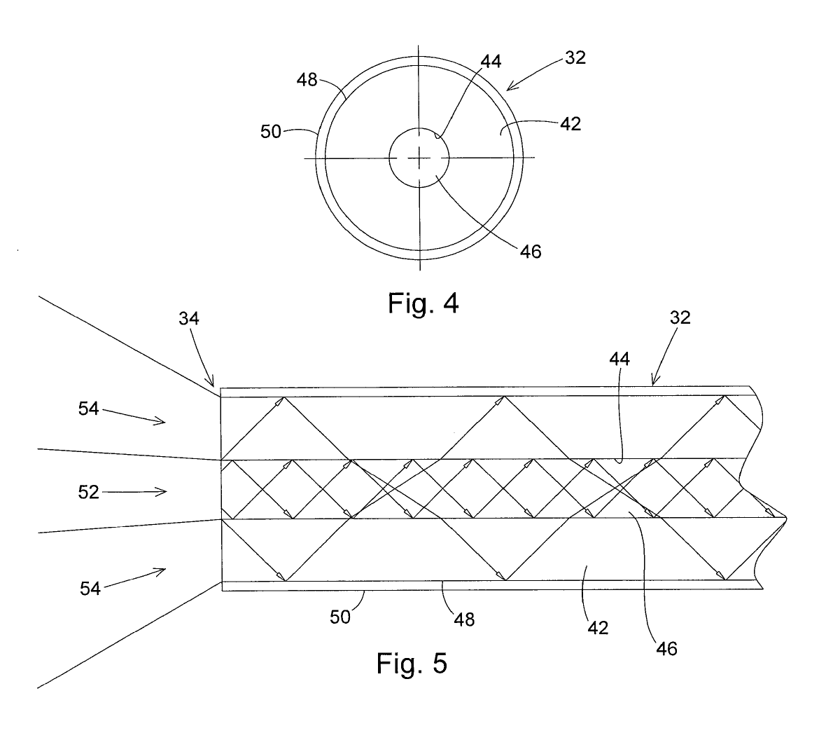

### DETAILED DESCRIPTION OF THE PREFERRED EMBODIMENTS

The surgical instrument of the invention is intended to provide both illumination light and laser light in laser eye surgery. However, it should be understood that the instrument of the invention may be used in other types of surgical procedures and that the instrument of the invention may be combined with other types of surgical instruments, for example, instruments that provide aspiration to a surgical site, or with a bipolar cautery device, or other types of surgical devices. The instrument can be designed as a disposable instrument, and can also be designed as a reusable instrument that is sterilized after each use.

The instrument has an elongate, narrow handle or hand piece 12 that has opposite proximal 14 and distal 16 ends. The handle 12 is dimensioned to a size similar to that of a pencil, to fit comfortably in the surgeon's hand and to be easily manually manipulated by the surgeon. A hollow interior bore 18 extends completely through the center of the handle 12 from the proximal end 14 to the distal end 16. In an alternative embodiment, the handle could be provided with a groove in the side of the handle that extends along the length of the handle.

An elongate, tubular tip 22 projects from the handle distal end 16. The tip is rigid and is preferably constructed from surgical steel. The tubular tip 22 has a hollow interior bore 24 that extends completely through the tip from a proximal end 26 of the tip to a distal end 28 of the tip. The tip proximal end 26 is received in the handle interior bore 18 at the handle distal end 16 and is secured to the handle. The tip bore 24 communicates with the handle bore 18 and has a center axis 30 that is coaxial with a center axis of the handle. The tip 22 projects axially from the handle distal end 16 to the distal end 28 of the tip. In the alternate embodiments of the instrument, the tip 22 can be curved along a portion of its length. In addition, in alternate embodiments the tip could be flexible, and the tip could be mounted to the handle for reciprocating movements of the tip into and out of the handle interior bore 18. In such an embodiment, an actuator would be provided on the handle for manipulation by the user's hand. The actuator would be connected with the tip to cause movements of the tip relative to the handle in response to movements of the actuator on the handle.

A dual core optic fiber 32 extends through the handle bore 18 and the tip bore 24. The optic fiber 32 has an elongate length that extends from a proximal end 34 to a distal end 36 of the optic fiber. A majority of the optic fiber length is outside of the instrument handle 12 and the instrument tip 22. The majority of the optic fiber length being outside of the instrument handle 12 allows the optic fiber length to flex freely as the instrument handle 12 is manipulated during use of the instrument. In the illustrated embodiment, the optic fiber 32 is secured stationary relative to the handle 12 and the tip 22 by adhesives or other equivalent means. In alternate embodiments of the invention, the tubular tip 22 could be movable relative to the optic fiber 32, or the optic fiber 32 could be movable through the handle interior bore 18 and the tip interior bore 24.

A laser light source connector 38 is provided at the optic fiber proximal end 34. The connector 38 is a conventional connector that is adapted for connecting the optic fiber proximal end 34 to a separate light source, preferably a light source that delivers coaxially aligned laser light and white light illumination. There are many different available light sources that are used in microsurgery and in particular ophthalmic surgery, and the connector 38 can be altered so that the instrument of the invention may be used with any of these available light sources. The proximal end 34 of the optic fiber 32 extends completely through the connector 38 and an end surface of the optic fiber proximal end 34 is positioned in the same plane as the proximal end of the connector 38. The optic fiber 32 extends from the connector 38 through the handle interior bore 18 and the tip interior bore 24 to the distal end 36 of the optic fiber positioned adjacent the tip distal end 28. The light received by the optic fiber proximal end 34 travels through the length of the optic fiber 32 and is emitted from the optic fiber distal end 36.

A novel feature of the invention is provided by the dual core construction of the optic fiber 32. The optic fiber 32 has four basic components that extend along the length of the fiber. The components include a tubular outer core 42 that has a hollow interior bore 44, an inner, center core 46 that extends through the interior bore 44 of the outer core 42, a cladding layer 48 that surrounds the outer core 42, and a buffer layer 50 that surrounds the cladding layer 48. The center core 46 extends the entire length of the optic fiber from the proximal end 34 to the distal end 36. The outer core 42 surrounds the center core 46 and also extends from the optic fiber proximal end 34 to the distal end 36. The interior bore 44 of the outer core 42 could also function as a passage that extends completely through the optic fiber 32. The passage could be employed for aspiration through the optic fiber, to deliver fluid through the optic fiber, or to accommodate other surgical devices such as a bipolar cautery device. The cladding layer 48 surrounds the outer core 42 and extends from the optic fiber proximal end 34 to the optic fiber distal end 36 in one embodiment, and to a position adjacent the distal end 36 in a further embodiment. The buffer layer 50 surrounds the cladding layer 48 and extends a majority of the length of the optic fiber from the connector 38 adjacent the proximal end 34 to the handle 12 adjacent the distal end 36. The two cores 42, 46 of the optic fiber are made of silica glass, each having a different index of refraction. Other materials that are capable of transmitting light may be used to construct the two cores 42, 46 other than silica glass. For example, the fibers could be constructed of a mixture of silica and plastic. Also, the inner core could be silica glass and the outer core could be plastic. In the dual core optic fiber 32, the outer core 42 functions as a first, illumination optic fiber that transmits illuminating light and the inner core 46 functions as a second, laser optic fiber that transmits laser light. In the preferred embodiment, the material of the first optic fiber 42 has a first index of refraction, and the material of the second optic fiber 46 has a second index of refraction. In the preferred embodiment where the second optic fiber 46 transmits laser light and the first optic fiber 42 transmits illumination light, the second index of refraction is larger than the first index of refraction. The outer core 42 has an index of refraction of 1.436 and the center core 46 has an index of refraction of 1.453 in the preferred embodiment. However, the index of refraction for the outer core could range between 1.26 and 1.59 and the index of refraction for the center core could range between 1.40 and 1.60.

The cladding layer 48 is made of a cladding material having a lower index of refraction than that of the outer core 42 and the center core 46. The cladding 48 has an index of refraction of 1.388 in the preferred embodiment, but the index of refraction could range between 1.25 and 1.40. The cladding could be constructed of silica glass or plastic or a mixture of both. Where the center core 46 and the outer core 42 transmit the light through the optic fiber 32, the cladding layer 48 keeps the light from exiting the dual cores. The cladding layer 48 works by having a lower refractive index from that of the dual cores 42, 46 such that, when the laser light hits the cladding layer 48 it is reflected back into the dual cores. Thus, the outer core 42 will always have an index of refraction between that of the inner core 46 and the cladding 48.

The buffer layer 50 protects the cladding layer 48 and the dual cores 42, 46 from damage.

The changes in the index of refraction between the center core 46, the outer core 42, and the cladding layer 48 give the center core 46 a lower Numerical Aperture (NA), and give the outer core 42 a much higher Numerical Aperture. This results in the center core 46 of the fiber only accepting light that has a narrow divergence angle, such as laser light, while the outer core 42 accepts light having a more divergent angle, such as the illuminating light from the source. This is depicted in FIG. 5 where the laser light 52 is shown entering the center core 46 and the illumination light 54 is shown entering the outer core 42 at the optic fiber proximal end 34.

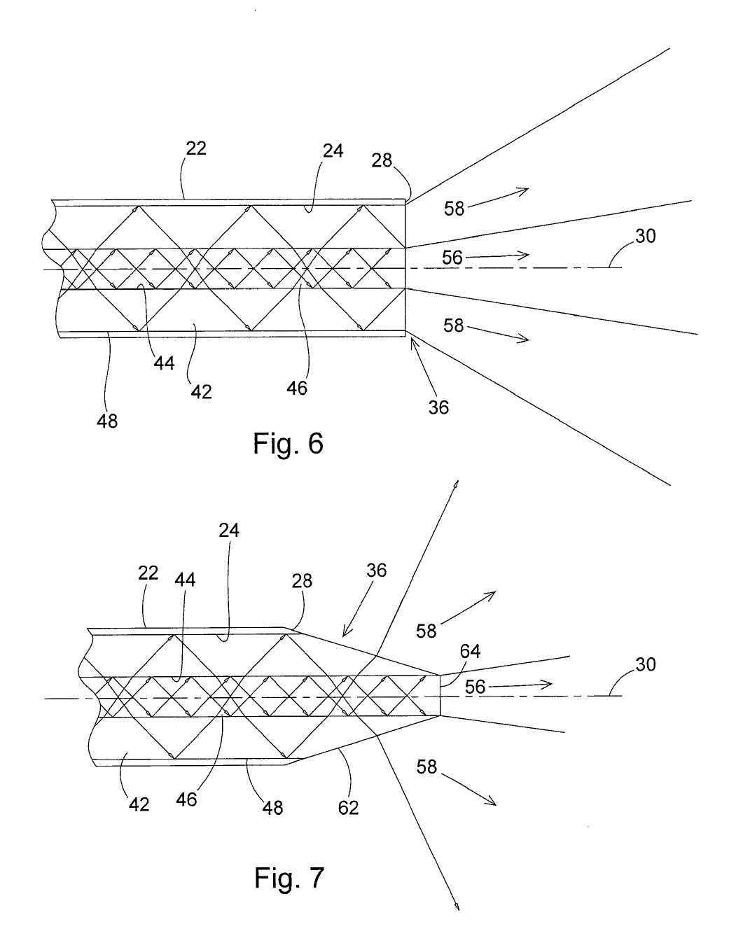

This relationship is also important at the optic fiber distal end 36 where the relationship is also true of the light leaving the optic fiber distal end. The center core 46 will emit a light having a narrow divergence angle 56 where the outer core 42 will emit a light having a larger divergence angle 58. FIG. 6 represents the laser light 56 and illumination light 58 emitted from the optic fiber distal end 56 where the distal end surface is normal to the center axis of the optic fiber. FIG. 7 represents the effective divergence angle of the outer core 42 being increased by tapering the distal end tip at the outer core 42, while leaving the distal end surface of the center core 46 normal to the optic fiber center axis. The tapered surface, preferably a beveled surface 62 at the distal end of the outer core 42 helps scatter the illumination light away from the optic fiber center axis 30, while the normal surface 64 at the distal end of the center core 46 remains unaffected. Other light diverging optics could be used at the distal end of the outer core 42 instead of the tapered surface 62. For best results the light emitted from the outer core 42 is given as high a Numerical Aperture as possible.

Thus, as discussed above, the surgical instrument of the invention provides a single microsurgical instrument that delivers both laser light and illumination light from a single instrument and from a single light source.

Although specific embodiments of the invention have been described herein, it should be understood that other modifications and variations may be made to the invention without departing from the protected concept of the invention.

1. A surgical instrument that provides light to a surgical site, the instrument comprising:
a handle that is dimensioned to be held in a hand and manually manipulated;
a tubular tip mounted on the handle and projecting from the handle to a distal end of the tip, the tip having an interior bore that extends through the tip to the tip distal end;
a light transmitting optic fiber, the optic fiber having a length that extends between opposite proximal and distal ends of the optic fiber, the optic fiber extending through the tip interior bore to the optic fiber distal end positioned adjacent the tip distal end, and the optic fiber having a hollow interior bore extending through at least a portion of the optic fiber to the optic fiber distal end.

2. The instrument of claim 1, further comprising:
the optic fiber having a center axis at the optic fiber distal end; and,
the optic fiber having light diverging optics at the optic fiber distal end that diverge light transmitted from the optic fiber distal end away from the optic fiber center axis.

3. The instrument of claim 2, further comprising:
the light diverging optics being a tapered surface on the optic fiber distal end.

4. The instrument of claim 3, further comprising:
the tapered surface being a conical surface that extends around the optic fiber hollow interior bore.

5. The instrument of claim 1, further comprising:
the optic fiber hollow interior bore extending through the optic fiber length and defining a passage through the optic fiber length from the optic fiber proximal end to the optic fiber distal end.

6. The instrument of claim 1, further comprising:
the optic fiber being secured stationary relative to the handle.

7. The instrument of claim 1, further comprising:
the tubular tip being a rigid tip that projects straight from the handle.

8. The instrument of claim 1, further comprising:
the optic fiber being constructed of silica glass.

9. The instrument of claim 1, further comprising:
the optic fiber being a first optic fiber; and,
a second light transmitting optic fiber, the second optic fiber having a length with opposite proximal and distal ends, the second optic fiber extending through the first optic fiber interior bore to the distal end of the second optic fiber positioned adjacent the distal end of the first optic fiber.

10. The instrument of claim 9, further comprising:
the first optic fiber being an illumination optic fiber that transmits illumination light and the second optic fiber being a laser optic fiber that transmits laser light.

11. The instrument of claim 9, further comprising:
a connector on both the first optic fiber proximal end and on the second optic fiber proximal end, the connector being adapted for connecting the first optic fiber proximal end and the second optic fiber proximal end to a light source.

12. The instrument of claim 9, further comprising:
the interior bore extending through the first optic fiber from the first optic fiber proximal end to the first optic fiber distal end; and,
the second optic fiber extending through the interior bore of the first optic fiber from the first optic fiber proximal end to the first optic fiber distal end.

13. The instrument of claim 12, further comprising:
the first optic fiber interior bore defining a passage along the first optic fiber.

14. The instrument of claim 9, further comprising:
the first optic fiber having a center axis at the first optic fiber distal end, and the first optic fiber having light diverging optics at the first optic fiber distal end that diverge light transmitted from the first optic fiber distal end away from the center axis.

15. The instrument of claim 14, further comprising:
the light diverging optics being a tapered surface on the first optic fiber distal end.

16. The instrument of claim 15, further comprising:
the second optic fiber having a distal end surface positioned in a plane that is perpendicular to the first optic fiber center axis.

17. The instrument of claim 9, further comprising:
the first optic fiber having a first index of refraction and the second optic fiber having a second index of refraction that is different from the first index of refraction.

18. The instrument of claim 17, further comprising:
the first index of refraction being smaller than the second index of refraction.

19. A surgical instrument that provides light to a surgical site, the instrument comprising:
a handle that is dimensioned to be manually manipulated;
a tubular tip mounted on the handle and projecting from the handle to a distal end of the tip, the tip having a hollow interior bore extending through the tip from the handle to the tip distal end;
a first light transmitting optic fiber having a length with opposite proximal and distal ends, the first optic fiber extending through the tip interior bore to the distal end of the first optic fiber positioned adjacent the tip distal end, the first optic fiber having a hollow interior bore extending through the length of the first optic fiber from the first optic fiber proximal end to the first optic fiber distal end; and,
a second light transmitting optic fiber having a length with opposite proximal and distal ends, the second optic fiber extending through the first optic fiber interior bore to the distal end of the second optic fiber positioned adjacent the distal end of the first optic fiber.

20. The instrument of claim 19, further comprising:
the first optic fiber and the second optic fiber having a common center axis at the distal ends of the first optic fiber and the second optic fiber; and,
the first optic fiber having light diverging optics at the distal end of the first optic fiber that diverge light transmitted from the first optic fiber distal end away from the center axis.

21. The instrument of claim 20, further comprising:
the light diverging optics on the first optic fiber distal end being a tapered surface on the first optic fiber distal end.

22. The instrument of claim 21, further comprising:
the second optic fiber having a distal end surface positioned in a plane that is perpendicular to the center axis.

23. The instrument of claim 19, further comprising:
the first optic fiber interior bore defining an interior passage through the first optic fiber length.

24. The instrument of claim 19, further comprising:
the first optic fiber and the second optic fiber being secured stationary relative to the handle.

25. The instrument of claim 19, further comprising:
the tuber tip being a rigid tip that projects straight from the handle.

26. The instrument of claim 19, further comprising:
the first optic fiber having a first index of refraction; and,
the second optic fiber having a second index of refraction that is different from the first index of refraction.

27. The instrument of claim 19, further comprising:
a connector on the first optic fiber proximal end and on the second optic fiber proximal end, the connector being adapted for connecting the first optic fiber proximal end and the second optic fiber proximal end to a light source.

28. The instrument of claim 27, further comprising:
the first optic fiber being an illumination optic fiber that transmits illumination light and the second optic fiber being a laser optic fiber that transmits laser light.

29. A surgical instrument that provides light to a surgical site, the instrument comprising:
a manually manipulable handle;
a tubular tip secured to the handle, the tip projecting from the handle to a distal end of the tip;
a first illumination light transmitting optic fiber having a length with opposite proximal and distal ends, the first illumination optic fiber extending through the handle and through the tip to the first illumination optic fiber distal end positioned adjacent the tip distal end;
a second laser light transmitting optic fiber having a length with opposite proximal and distal ends, the second laser optic fiber extending through a center of the first illumination optic fiber, the second laser optic fiber extending through the center of the first illumination optic fiber from the proximal end of the first illumination optic fiber to the distal end of the first illumination optic fiber;
the second laser optic fiber having an index of refraction and the first illumination optic fiber having an index of refraction, the second laser optic fiber index of refraction being larger than the first illumination optic fiber index of refraction.

30. The instrument of claim 29, further comprising:
the first optic fiber and the second optic fiber having a common center axis at the distal ends of the first optic fiber and the second optic fiber; and,
the first optic fiber having light diverging optics at the distal end of the first optic fiber that diverge light transmitted from the first optic fiber distal end away from the center axis.

31. The instrument of claim 30, further comprising:
the light diverging optics on the first optic fiber distal end being a tapered surface on the first optic fiber distal end.

32. The instrument of claim 31, further comprising:
the second optic fiber having a distal end surface positioned in a plane that is perpendicular to the center axis.

33. The instrument of claim 29, further comprising:
the first optic fiber and the second optic fiber being secured stationary relative to the handle.

34. The instrument of claim 29, further comprising:
the tip being a rigid tip that projects straight from the handle.

35. The instrument of claim 29, further comprising:
the first optic fiber being constructed of silica glass; and,
the second optic fiber being constructed of silica glass.

36. The instrument of claim 29, further comprising:
a connector on the first optic fiber proximal end and on the second optic fiber proximal end, the connector being adapted for connecting the first optic fiber proximal end and the second optic fiber proximal end to a light source.
